(19) United States
(12) Patent Application Publication
(10) Pub. No.: US 2008/0234809 A1
(43) Pub. Date: Sep. 25, 2008

(54) Stent Graft System With Injection Tube

(75) Inventor: Trevor Greenan, Santa Rosa, CA (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 11/690,186

(22) Filed: Mar. 23, 2007

(51) Int. Cl. A61F 2/06

(52) U.S. Cl. 623/1.42

(57) ABSTRACT

A stent graft system with injection tube, the stent graft system for injecting an agent into an aneurysmal sac from an external pressurized source, including a stent graft having a permeable graft and a framework supporting the permeable graft; and an injection tube connected to the stent graft adjacent to the permeable graft, the injection tube having a plurality of holes adjacent to the permeable graft. The external pressurized source forces the agent through the plurality of holes into the aneurysmal sac.

### TECHNICAL FIELD

The technical field of this disclosure is medical implantation devices, particularly, a stent graft system with an injection tube.

### BACKGROUND OF THE INVENTION

Stent grafts have been developed for the treatment of abdominal aortic aneurysms. An abdominal aortic aneurysm is a sac that forms in the wall of the abdominal aorta, which is the main vessel of the arterial system of the body that extends through the abdomen. Abdominal aortic aneurysms can lose elasticity over time and rupture under normal blood pressure. A stent graft is a woven tube (graft) supported by a tubular metal stent. The stent graft is placed inside of an aneurysmal vessel to exclude the abdominal aortic aneurysm from normal blood flow and reduce pressure on the aneurysmal vessel. Stent grafts employ sealing regions at the proximal and distal ends to seal the stent graft to the normal aortic wall and prevent blood flow between the stent graft and the aneurysmal vessel.

There is the opportunity to provide additional therapy during insertion of a stent graft by injection of therapeutic agents into the aneurysmal sac. The physician has local access to the aneurysmal sac while placing the stent graft. Presently, a catheter is inserted into the aneurysmal sac during the procedure and the therapeutic agent injected at a single point within the aneurysmal sac. This approach only provides the therapeutic agent to a small area, which may not be the area where the therapeutic agent is most effective. The therapeutic agent can form a large pocket at the catheter tip, rather than being evenly distributed relative to the aneurysmal sac and the stent graft. For example, a therapeutic agent that is most effective in the sealing region may not reach the sealing region. In addition, the placement of the catheter tip is uncertain and makes it difficult for the physician to precisely locate where they want to inject the therapeutic agent.

U.S. Pat. No. 6,355,063 to Calcotte discloses an improved ePTFE-based delivery graft intended to dispense a bioactive agent such as a drug into the blood stream. Spiral hollow tubing is wrapped in a helical fashion around, or otherwise brought into contact with an outer wall of a porous ePTFE graft and adhered thereto. The agent is delivered to the lumen of the graft by infusing the agent through the porous interstices of the graft wall. Thus, the bioactive agent is conducted by the hollow tubing from a source to the outer surface of an ePTFE graft where it is released to diffuse into the graft.

U.S. Patent Application Publication Number US 2003/0074048 to Sherry discloses a tubular prosthesis, which includes a tubular member (stent or stent/graft combination) and an outer covering having portions sealed to the tubular member. The tubular member is impervious to a pre-determined fluid, particularly an occluding fluid, while the outer cover is pervious to the pre-determined fluid.

U.S. Patent Application Publication Number US 2005/0171593 to Whirley, et al., discloses inflatable porous implants, such as grafts, stent-grafts, and bladders, providing for direct delivery of larger, more precise dosages of drugs over longer administration periods into the body. The implants further provide a mechanical or structural function in addition to drug delivery in a single integrated structure.

It would be desirable to have a stent graft system with an injection tube that would overcome the above disadvantages.

### SUMMARY OF THE INVENTION

One aspect according to the present invention provides a stent graft system for injecting an agent into an aneurysmal sac from an external pressurized source, including a stent graft having a permeable graft and a framework supporting the permeable graft; and an injection tube connected to the stent graft adjacent to the permeable graft, the injection tube having a plurality of holes adjacent to the permeable graft. The external pressurized source forces the agent through the plurality of holes into the aneurysmal sac.

Another aspect according to the present invention provides a stent graft system for injecting an agent into an aneurysmal sac from an external pressurized source, including a stent graft having a permeable graft and a framework supporting the permeable graft; and means for injecting the agent from the external pressurized source into the aneurysmal sac adjacent to the permeable graft. The injecting means is connected to and disposed on the stent graft.

Another aspect according to the present invention provides a method of injecting an agent into an aneurysmal sac from an external pressurized source, including providing a stent graft having an injection tube with a plurality of holes, a permeable graft, and a framework supporting the permeable graft, the injection tube being connected to the stent graft adjacent to the permeable graft and the plurality of holes being adjacent to the permeable graft; inserting the stent graft into the aneurysmal sac in a compressed state; deploying the stent graft to an expanded state; and forcing the agent from the external pressurized source through the plurality of holes into the aneurysmal sac.

The foregoing and other features and advantages will become further apparent from the following detailed description of the presently preferred embodiments, read in conjunction with the accompanying drawings

### BRIEF DESCRIPTION OF THE DRAWINGS

FIG. 1 is a side view of a stent graft system with an injection tube made in accordance with the present invention;

FIGS. 2A-2B are a detail view and radial cross section view, respectively, of a stent graft system with an injection tube made in accordance with the present invention;

FIG. 3 is a side view of another stent graft system with an injection tube made in accordance with the present invention;

FIG. 4 is a side view of another stent graft system with an injection tube made in accordance with the present invention; and

FIG. 5 is a flowchart of method of injecting an agent into an aneurysmal sac using a stent graft system with an injection tube made in accordance with the present invention.

### DETAILED DESCRIPTION

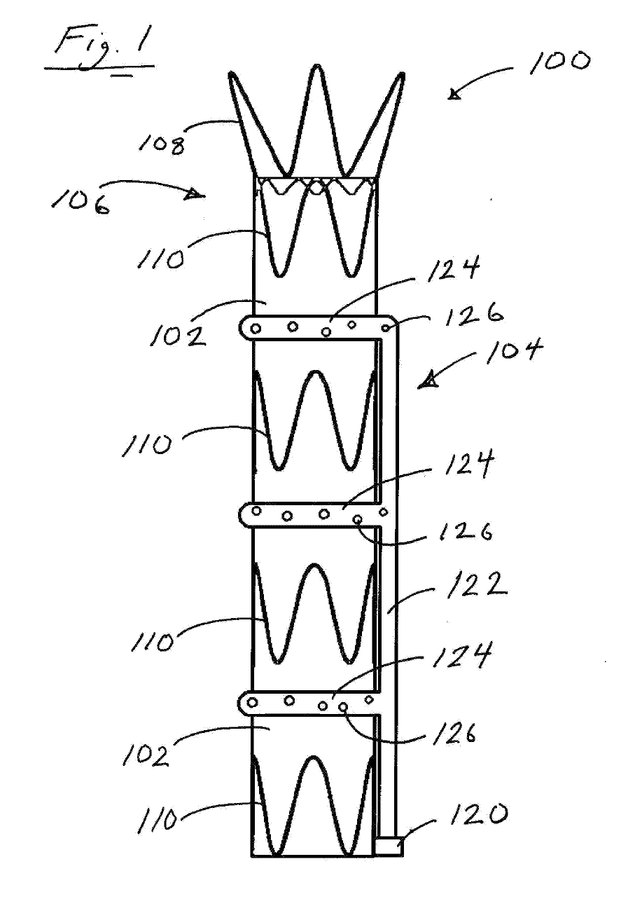

FIG. 1 is a side view of a stent graft system with an injection tube made in accordance with the present invention. The stent graft system 100 includes a stent graft 106 and an injection tube 104 connected to the stent graft 106. The stent graft 106 includes a permeable graft 102 and a framework of stent rings 108, 110 supporting the permeable graft 102. In this example, the injection tube 104 is disposed adjacent the permeable graft 102 and includes an injection connector 120, an injection manifold 122, and one or more injection rings 124. The injection rings 124 include holes 126 adjacent the permeable graft 102. In operation, an external pressurized source (not shown), such as a syringe or a pump, releasably connected to the injection connector 120 forces an agent through the injection tube 104 and out the holes 126 into the aneurysmal sac. The injection tube 104 allows injection of an agent, such as a therapeutic agent or an indicating agent, directly into the aneurysmal sac adjacent the permeable graft 102.

The injection tube 104 can be fabricated as a unit and secured to the stent graft 106 with an adhesive, stitching, or the like. In one embodiment, the injection tube 104 is a thin walled tube that collapses unless the external pressurized source is providing pressure to the agent in the injection tube 104 and the agent is flowing through the holes 126. In another embodiment, the injection tube 104 maintains an open lumen even when the external pressurized source is not providing pressure. The cross section of the injection tube 104 can be round, semicircular, elliptical, square, rectangular, or any other cross section as desired for a particular application. In this example, the injection rings 124 are uniformly distributed along the axial length of the stent graft 106. In another embodiment, the one or more injection rings can be located in a particular area of the stent graft 106, such as at the proximal and/or distal sealing region. In this example, the holes 126 are located in the injection rings 124. In another embodiment, the manifold 122 can include holes. The injection tube 104 can be of biocompatible materials, such as expanded porous polytetrafluoroethylene (EPTFE), polyurethane, nylon, or the like. The holes 126 can be from 0.1 to 5 mm in diameter, such as from 0.1 to 5 mm, or from 0.25 to 3 mm.

The holes 126 are sized so that the external pressurized source forces the agent out the holes 126 into the aneurysmal sac. The pressure just upstream of the holes 126 is a low pressure, i.e., a pressure only slightly above the pressure in the aneurysmal sac. Those skilled in the art will appreciate that the hole size and pressure required at the external pressurized source can be selected in light of the viscosity of the agent to be injected into the aneurysmal sac. The injection tube 104 can incorporate flow regulating features to provide even flow from all the holes 126. In one embodiment, the size of the holes can vary with smaller diameter holes in the higher pressure regions of the injection tube 104 and larger diameter holes in the lower pressure regions. In another embodiment, the number of holes of the same diameter can vary, with fewer holes in the higher pressure regions of the injection tube 104. In yet another embodiment, the cross section of the injection tube 104 can vary with distance from the injection connector 120. For example, the injection manifold 122 and/or the injection rings 124 can have a smaller flow cross section near the injection connector 120. In another example, the injection rings 124 nearer the injection connector 120 can have a smaller flow cross section than the injection rings 124 further away from the injection connector 120. In yet another embodiment, the flow cross section of the injection tube 104 can include flow restrictors to provide even flow from all the holes 126.

The injection tube 104 can be any tubing disposed in any pattern adjacent the permeable graft 102 with holes 126 allowing injection of an agent into the aneurysmal sac adjacent to the permeable graft 102. The framework of the stent graft 106 supports the permeable graft, so the injection tube 104 can be flexible and need not provide support. In this example, the injection rings 124 are joined by the injection manifold 122 in a manifold-ring pattern. The injection rings 124 can completely or partially encircle the stent graft 106. In another embodiment, the injection tube 104 is a tube wound helically about the axial length of the stent graft 106 in a helical pattern. In yet another embodiment, the injection tube 104 is a tube disposed in a sinusoidal pattern back and forth along at least part of the axial length of the stent graft 106. In yet another embodiment, the injection tube 104 is two or more rings about the perimeter of the stent graft 106 with axially-aligned tubes connecting the rings in a hoop-and-bar pattern. In any embodiment, the holes can be located of the injection tube 104 to provide the agent to the region adjacent the permeable graft as desired.

The injection connector 120 can be any connector allowing disconnection of the injection tube 104 from the supply catheter attached to the external pressurized source. In one embodiment, the injection connector 120 is a tube sized to be slideably received in the supply catheter with at least one O-ring on the injection connector and/or the supply catheter sealing the connection. The connection is separated after the procedure by pulling the supply catheter from the tube. In another embodiment, the supply catheter is sized to be slideably received in the tube. In yet another embodiment, the injection connector 120 is a continuous length of tubing with the supply catheter continuing into the injection tube 104. The continuous length of tubing is scored to provide the injection connector 120. The scored portion fails, disconnecting the supply catheter from the injection tube 104.

Those skilled in the art will appreciate that the stent graft 106 can be any stent graft with a framework. The permeable graft 102 can be any made of any biocompatible material, such as Dacron™ or expanded porous polytetrafluoroethylene (EPTFE). The framework can be any framework suitable for supporting the permeable graft. In this example, the framework is a number of stent rings including a proximal stent ring 108 and body stent rings 110 operably connected about the permeable graft 102. Proximal and distal are defined relative to the fluid flow in the lumen in which the stent graft is installed, with the flow being from proximal to distal. The number and axial distance between stent rings can be selected as desired for a particular application. In another embodiment, the framework includes a distal stent ring. In yet another embodiment, the framework includes a number of stent rings joined into a unitized stent ring, such as a continuous diamond pattern. In one embodiment, the stent graft is a branching stent graft having a body and branches. Each branch can have its own framework separate from the framework for the body.

The stent graft is typically delivered to the site of the aortic aneurysm in a compressed state and deployed to an expanded state. In one embodiment, the framework of the stent graft is made of a deformable material that can be expanded with a balloon catheter, such as a balloon catheter used in percutaneous transluminal coronary angioplasty (PTCA). In another embodiment, the framework of the stent graft is made of a shape memory alloy, such as nitinol, that expands the framework to a predetermined shape when the stent rings are exposed to body temperature.

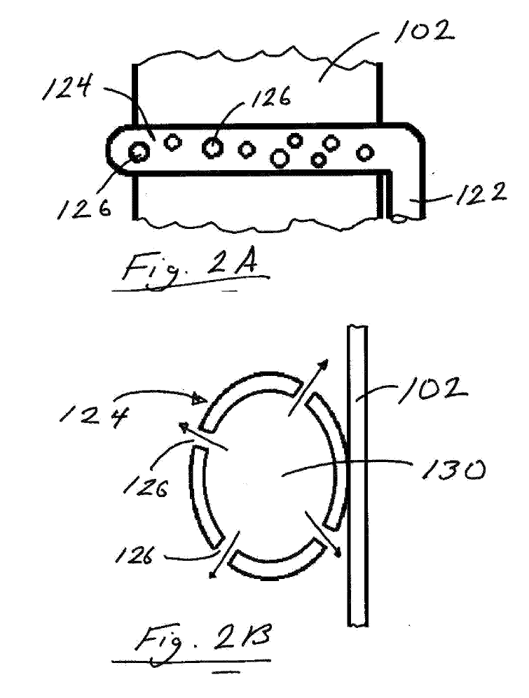

FIGS. 2A-2B, in which like elements share like reference numbers with each other and with FIG. 1, are a detail view and radial cross section view, respectively, of a stent graft system with an injection tube made in accordance with the present invention. In this example, the injection ring 124 attached to the injection manifold 122 includes a number of holes 126. As illustrated by the arrows through the holes 126 from the lumen 130 in FIG. 2B, the agent exits the holes 126 and enters the aneurysmal sac adjacent the permeable graft 102. The cross section of the injection ring 124 can be round, semicircular, elliptical, square, rectangular, or any other cross section as desired for a particular application. The injection ring 124 can be of biocompatible materials, such as expanded porous polytetrafluoroethylene (EPTFE), polyurethane, nylon, or the like. The holes 126 can be distributed in the injection ring 124 in any pattern desired for a particular application.

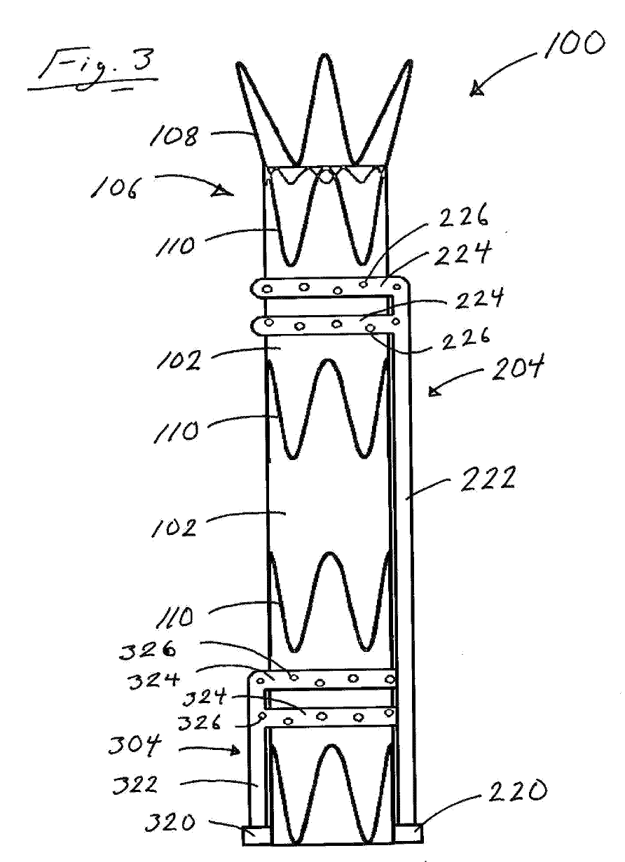

FIG. 3, in which like elements share like reference numbers with FIG. 1, is a side view of another stent graft system with an injection tube made in accordance with the present invention. In this embodiment, the stent graft system 100 includes a stent graft 106, a first injection tube 204, and second injection tube 304. The first injection tube 204 and second injection tube 304 are independent so that different agents can be provided by each, a single agent can be provided by each at different pressures, or different agents can be provided by each at different pressures.

The first injection tube 204 includes an injection connector 220, an injection manifold 222, and one or more injection rings 224. The second injection tube 304 includes an injection connector 320, an injection manifold 322, and one or more injection rings 324. In one embodiment, the stent graft system 100 is connected to two external pressurized sources (not shown) with two different catheters at the injection connectors 220 and 320. In another embodiment, the stent graft system 100 is connected to two external pressurized sources (not shown) with a single catheter having two lumens with two catheter connectors. Each of the catheter connectors is connected to one of the injection connectors 220 and 320. In yet another embodiment, the stent graft system 100 is connected to two external pressurized sources (not shown) with a single catheter having a single catheter connector. The injection connectors 220 and 320 are combined into a single injection connector and the branching into the first injection tube 204 and the second injection tube 304 occurs downstream of the single injection connector.

Those skilled in the art will appreciate that any number of injection rings can be provided for each injection tube and that the injection rings can be distributed along the stent graft as desired for a particular application. In addition, any number of independent injection tubes can be provided on the stent graft. The pattern of the injection tube on the stent graft is not limited to the manifold pattern illustrated in FIG. 3: the injection tube pattern can be any pattern desired for a particular application, such as a manifold-ring pattern, a helical pattern, a sinusoidal pattern, a hoop-and-bar pattern, or the like.

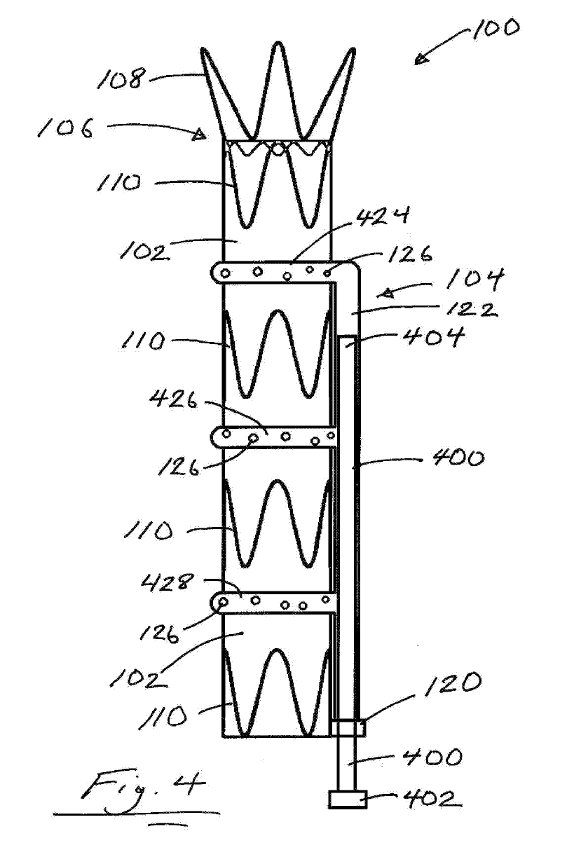

FIG. 4, in which like elements share like reference numbers with FIG. 1, is a side view of another stent graft system with an injection tube made in accordance with the present invention. In this example, a feed tube within the injection tube provides the agent to the injection tube. The feed tube 400 is slidably received within the injection tube 104 and connected to the external pressurized source (not shown) with a catheter attached to feed tube connection 402. The feed tube 400 is illustrated with a solid line for clarity of illustration. Another catheter is attached to the injection connector 120 around the feed tube 400 to provide a path for the feed tube 400 between the outside of the patient and the injection tube 104.

In operation, the feed tube 400 is advanced through the injection manifold 122 until the feed tube tip 404 is near the proximal injection ring 424. The external pressurized source injects the agent, which flows from the holes 126 of the proximal injection ring 424 into the aneurysmal sac. The feed tube 400 is drawn back in the injection manifold 122 until the feed tube tip 404 is near the medial injection ring 426. The external pressurized source injects the agent, which flows primarily from the holes 126 of the medial injection ring 426 into the aneurysmal sac. The feed tube 400 is drawn back in the injection manifold 122 again until the feed tube tip 404 is near the distal injection ring 428. The external pressurized source injects the agent, which flows primarily from the holes 126 of the distal injection ring 428 into the aneurysmal sac. The feed tube 400 can then be withdrawn from the patient. Those skilled in the art will appreciate that the injection sequence can be selected as desired for a particular application. In one example, the first injection could be at the distal injection ring 428 and the feed tube 400 advanced into the injection tube 104 for the next injection. In another example, the injection and the movement of the feed tube 400 in the injection tube 104 can be performed simultaneously.

Various configurations of the feed tube 400 can be selected as desired for a particular purpose. In one embodiment, the end of the feed tube tip 404 is open. In another embodiment, the end of the feed tube tip 404 is closed and a port is provided in the side of the feed tube tip 404, so that the agent flows out of the side of the feed tube 400. In yet another embodiment, the outer diameter of the feed tube 400 is slightly smaller than the inner diameter of the injection tube 104, so that little fluid leaks between the two. Those skilled in the art will appreciate that the feed tube configuration can be selected as desired to match the injection tube pattern, such as a manifold-ring pattern, a helical pattern, a sinusoidal pattern, a hoop-and-bar pattern, or the like.

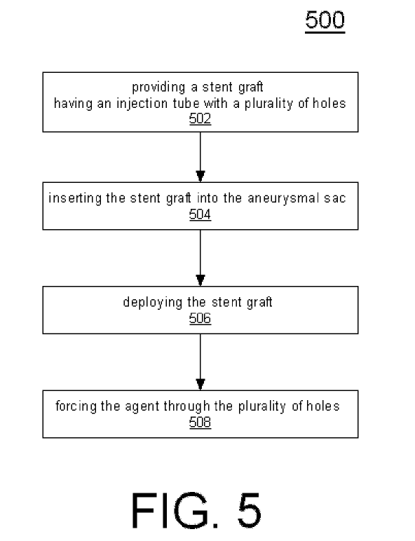

FIG. 5 is a flowchart of method of injecting an agent into an aneurysmal sac using a stent graft system with an injection tube made in accordance with the present invention. The method 500 includes providing a stent graft having injection tube having a plurality of holes 502, inserting the stent graft into the aneurysmal sac 504, deploying the stent graft 506, and forcing the agent through the number of holes 508.

The method 500 injects an agent into an aneurysmal sac from an external pressurized source. The agent can be any agent useful for therapy and/or imaging. Particular examples of agents include therapeutic agents, drugs, platelet poor plasma, saline with microspheres, thrombin, growth factors, platelet rich plasma, adhesives such as fibrin or cyanoacrylates, bulk filler, combinations thereof, and the like. Certain agents, such as growth factors, platelet rich plasma, adhesives such as fibrin or cyanoacrylates, and bulk fillers, can be injected into the aneurysmal sac near the sealing regions to seal the stent graft to the normal aortic wall and prevent blood flow between the stent graft and the aneurysmal vessel. The agent can include radionuclides for therapeutic or imaging. The agent can include radiopaque materials for imaging. The agent can be any material that will flow under pressure, such as liquids, slurries, gels, and the like.

The injection can be into the aneurysmal sac, such as the aneurysmal sac of an abdominal aortic aneurysm. Those skilled in the art will appreciate that the stent graft system can be used with any aneurysm in the body and is not limited to use with abdominal aortic aneurysms. The external pressurized source can be any pressurized source external to the patient pressurizing the agent to flow through a catheter to the injection tube and out the holes of the injection tube into the aneurysmal sac. Examples of external pressurized sources include syringes and pumps.

The step of providing a stent graft having injection tube having a plurality of holes 502 includes providing a stent graft having an injection tube with a number of holes, a permeable graft, and a framework supporting the permeable graft. The injection tube is connected to the stent graft adjacent to the permeable graft and the holes are adjacent to the permeable graft. The holes are sized to permit ready flow of the agent from the lumen of the injection tube. In one embodiment, the holes have a diameter greater than 0.1 mm, such as between 0.1 and 5 mm, or between 0.25 and 3 mm.

The inserting the stent graft into the aneurysmal sac 504 includes inserting the stent graft into the aneurysmal sac in a compressed state. For the example of an abdominal aortic aneurysm, a catheter is advanced to the abdominal aortic aneurysm through the femoral artery, the carotid artery, or the subclavian artery. The catheter is guided to the location of the aneurysm with X-ray or fluoroscopic data and the stent graft advanced to the aneurysm through the catheter. The stent graft in the compressed state has a smaller diameter than the stent graft in the expanded state so that the stent graft can pass through the catheter to the aneurysmal sac.

The deploying the stent graft 506 includes deploying the stent graft to an expanded state. When the stent graft has reached the abdominal aortic aneurysm and is outside the catheter, the stent graft can be allowed to expand or expanded. In one embodiment, the framework of the stent graft is made of a shape memory alloy, such as nitinol, that expands the stent graft to a predetermined shape when the framework is exposed to body temperature. In another embodiment, the framework of the stent graft are made of elastic alloy and held compressed with dissolvable ties. The dissolvable ties dissolve and the stent graft expands when the dissolvable ties are exposed to the fluid in the vessel. In another embodiment, the framework of the stent graft are made of deformable alloy and expanded with a balloon, such as a balloon used in percutaneous transluminal coronary angioplasty (PTCA).

The forcing the agent through the plurality of holes 508 includes forcing the agent from the external pressurized source through the number of holes into the aneurysmal sac. In one embodiment, the flow from the holes is uniform over the length of the injection tube. In another embodiment, the flow from the holes is greater in a portion of the length of the injection tube.

The forcing the agent through the plurality of holes 508 can include forcing the agent through a feed tube slidably disposed in the injection tube and out the holes. In one embodiment, the feed tube can be stopped at particular locations along the length of the injection tube and the agent can be injected at the particular locations. The particular locations can be selected so that the agent is most effective at the particular location and/or the amount injected at the particular location is most effective. In another embodiment, the feed tube can be kept moving along the length of the injection tube and the agent injected as the feed tube moves.

After the agent is in the aneurysmal sac, the agent can interact with the thrombus in the aneurysmal sac and/or the permeable graft. In one embodiment, the agent mixes with the thrombus to form a thrombus product. The thrombus product can have a beneficial characteristic, such as providing a protective layer. The thrombus product can interact with the permeable graft, such as attaching the thrombus product on and/or in the permeable graft.

The injection can be performed with a mixture of agents as required for a particular application. The injection can also be performed sequentially with a number of different agents. After all the desired injections have been performed, the catheter can be detached from the injection tube and removed from the patient.

While specific embodiments of the invention are disclosed herein, various changes and modifications can be made without departing from the spirit and scope of the invention. The scope of the invention is indicated in the appended claims, and all changes that come within the meaning and range of equivalents are intended to be embraced therein.

1. A stent graft system for injecting an agent into an aneurysmal sac from an external pressurized source comprising:
a stent graft having a permeable graft and a framework supporting the permeable graft; and
an injection tube connected to the stent graft adjacent to the permeable graft, the injection tube having a plurality of holes adjacent to the permeable graft;
wherein the external pressurized source forces the agent through the plurality of holes into the aneurysmal sac.

2. The system of claim 1 wherein the agent is selected from the group consisting of therapeutic agents, drugs, platelet poor plasma, saline with microspheres, thrombin, growth factors, platelet rich plasma, adhesives, fibrin, cyanoacrylates, bulk filler, radionuclides, radiopaque materials, and combinations thereof.

3. The system of claim 1 wherein the external pressurized source is selected from the group consisting of a syringe and a pump.

4. The system of claim 1 wherein the framework is selected from the group consisting of stet rings and a unitized stent ring.

5. The system of claim 1 wherein the injection tube is disposed on the stent ring in a pattern to provide a greater number of the plurality of holes at a region of the stent graft.

6. The system of claim 5 wherein the region is a sealing region.

7. The system of claim 1 wherein the injection tube is disposed on the stent ring in a pattern selected from the group consisting of a manifold-ring pattern, a helical pattern, a sinusoidal pattern, and a hoop-and-bar pattern.

8. The system of claim 1 wherein injection tube collapses when the agent is not flowing through the plurality of holes.

9. The system of claim 1 wherein each of the plurality of holes has a diameter between 0.1 and 5 mm.

10. The system of claim 1 wherein each of the plurality of holes has a diameter between 0.25 and 3 mm.

11. The system of claim 1 wherein the plurality of holes are distributed along the injection tube to provide uniform flow from the plurality of holes.

12. The system of claim 1 wherein the plurality of holes are distributed along the injection tube to provide greater flow from the plurality of holes in a portion of the injection tube.

13. The system of claim 1 wherein the injection tube has an inner diameter and the inner diameter varies along the injection tube to provide uniform flow from the plurality of holes.

14. The system of claim 1 wherein the injection tube has an inner diameter and the inner diameter varies along the injection tube to provide greater flow from the plurality of holes in a portion of the injection tube.

15. The system of claim 1 wherein the injection tube has a lumen and at least one flow restrictor disposed in the lumen.

16. The system of claim 1 further comprising a second injection tube connected to the stent graft adjacent to the permeable graft, the second injection tube having a second plurality of holes adjacent to the permeable graft.

17. The system of claim 1 further comprising a feed tube slidably disposed in the injection tube.

18. A stent graft system for injecting an agent into an aneurysmal sac from an external pressurized source comprising:
a stent graft having a permeable graft and a framework supporting the permeable graft; and
means for injecting the agent from the external pressurized source into the aneurysmal sac adjacent to the permeable graft;
wherein the injecting means is connected to and disposed on the stent graft.

19. The system of claim 18 further comprising means for providing uniform flow from the injecting means.

20. The system of claim 18 further comprising means for providing greater regional flow from the injecting means.

21. The system of claim 18 further comprising means for feeding the agent into the injecting means, the feeding means being slidably disposed in the injecting means.

22. A method of injecting an agent into an aneurysmal sac from an external pressurized source comprising:
providing a stent graft having an injection tube with a plurality of holes, a permeable graft, and a framework supporting the permeable graft, the injection tube being connected to the stent graft adjacent to the permeable graft and the plurality of holes being adjacent to the permeable graft;
inserting the stent graft into the aneurysmal sac in a compressed state;
deploying the stent graft to an expanded state; and
forcing the agent from the external pressurized source through the plurality of holes into the aneurysmal sac.

23. The method of claim 22 wherein the agent is selected from the group consisting of therapeutic agents, drugs, platelet poor plasma, saline with microspheres, thrombin, growth factors, platelet rich plasma, adhesives, fibrin, cyanoacrylates, bulk filler, radionuclides, radiopaque materials, and combinations thereof.

24. The method of claim 22 wherein each of the plurality of holes has a diameter between 0.1 and 5 mm.

25. The method of claim 22 wherein each of the plurality of holes has a diameter between 0.25 and 3 mm.

26. The method of claim 22 wherein the forcing comprises forcing the agent through a feed tube slidably disposed in the injection tube and out the plurality of holes.

27. The method of claim 22 wherein the aneurysmal sac contains thrombus, the thrombus and the agent forming a thrombus product.

28. The method of claim 22 wherein the thrombus product interacts with the permeable graft.

29. The method of claim 22 further comprising forcing a second agent from the external pressurized source through the plurality of holes into the aneurysmal sac.

30. The method of claim 22 wherein the stent graft has a second injection tube connected to the stent graft adjacent to the permeable graft, the second injection tube having a second plurality of holes adjacent to the permeable graft, and further comprising forcing a second agent from the external pressurized source through the second plurality of holes into the aneurysmal sac.
